(19) United States
(12) Patent Application Publication
(10) Pub. No.: US 2023/0012507 A1
(43) Pub. Date: Jan. 19, 2023

(54) WEARABLE SWEAT SENSOR

(71) Applicant: NATIONAL UNIVERSITY OF SINGAPORE, Singapore (SG)

(72) Inventors: Ananta Narayanan BALAJI, Singapore (SG); Yuan CHEN, Singapore (SG); Bo WANG, Singapore (SG); Li-Shiuan PEH, Singapore (SG); Huilin SHAO, Singapore (SG)

(21) Appl. No.: 17/780,357

(22) PCT Filed: Nov. 25, 2020
(86) PCT No.: PCT/SG2020/050689

§ 371 Date: May 26, 2022

(30) Foreign Application Priority Data
Nov. 29, 2019 (SG) 10201911412T

(51) Int. Cl. A61B 5/145; A61B 5/0205; A61B 5/1455; A61B 5/00; A61B 5/1477; G01N 21/80

(52) U.S. Cl. CPC A61B 5/14517; A61B 5/0205; A61B 5/14532; A61B 5/14546; A61B 5/14552; A61B 5/6801; A61B 5/14539; A61B 5/1477; G01N 21/80; A61B 5/02433

(57) ABSTRACT

A wearable sweat sensor for detecting one or more analytes in human sweat comprises an optical module comprising at least one light source and at least one light detector; at least one sensor layer optically coupled to the optical module and having optical absorbance properties that are dependent on the concentration of a target analyte of said one or more analytes; and one or more processors in communication with the optical module. The one or more processors are configured to: cause light from the at least one light source to be transmitted towards, and/or through, the at least one sensor layer; obtain, from the at least one light detector, one or more optical signals reflected and/or transmitted from the at least one sensor layer; and determine, from at least one wavelength component of the one or more optical signals, a target analyte concentration.

### TECHNICAL FIELD

The present disclosure relates to a wearable sweat sensor.

### BACKGROUND

Most present day fitness trackers and smart watches measure critical health indicators such as heart rate, SpO₂ concentration, sleep cycle etc., but do not track health indicators at the molecular level. Accordingly, various attempts have been made to develop chemical sensors which can non-invasively measure analytes available in raw biofluids such as sweat, tears and urine. Of all the available raw biofluids, sweat is the most readily and non-intrusively obtained, and thus is the most suitable choice for continuous real-time monitoring of indicators at the molecular level. In addition, sweat contains a rich amount of biomarkers such as sodium (Na⁺), chloride (Cl⁻), potassium (K⁺), calcium (Ca²⁺), pH, glucose, lactate and the like.

Amongst the detectable bio-markers in sweat, pH plays a very important role in the diagnosis of many critical health conditions. Variations in pH value of skin can aid in the diagnosis of skin conditions such as dermatitis, acne and other skin infections. It is also notable that the sweat of a dehydrated individual will show an increase in concentration of Na⁺, and a proxy indicator for this increase is an increase in pH value, since the higher the sodium (Na+) levels, the higher the pH of sweat will be. Also, an increase in perspiration rate triggers a rise in the pH value of sweat. The pH value of sweat therefore provides information regarding hydration level, which is important for fitness tracking as well as for diagnosing certain medical conditions.

A wearable device provides a natural platform for real-time continuous sensing of sweat, as it is in constant contact with human skin. As for existing fitness trackers that sense human heart rate continuously, repeatedly reusing the same low-cost wearable, sweat sensing on wearables should also be achievable on a low-cost, reusable wearable platform.

Previous wearable devices for real-time continuous monitoring of pH in sweat include those that incorporate colorimetric sensors which measure colour changes in treated fabrics or in colorimetric reagents contained in microfluidic channels. However, these have a short lifetime or are non-reusable due to the need to replace reagents.

Additionally, they cannot track time dependent changes in the concentration of biomarkers in sweat and hence are unsuitable for real-time continuous sensing of pH in sweat.

Other devices incorporate electrochemical sensors. However, these devices either have low long-term stability (of the order of 10 days) or are highly complex to manufacture, and require expensive instrumentation.

A further proposed sweat sensor has used a pH sensitive dye with paired emitter-detector red LEDs for continuous pH sensing. However, the pH sensitive dye is not reusable, and the sensor requires complex pumping methods to pump sweat into the pH sensor.

It is desirable therefore to provide a sweat sensor that addresses one or more of the above difficulties, or at least provides a useful alternative.

### SUMMARY

Disclosed herein is a wearable sweat sensor for detecting one or more analytes in human sweat, comprising:
an optical module comprising at least one light source and at least one light detector attached to a support;
at least one sensor layer optically coupled to the optical module, the at least one sensor layer having optical absorbance properties that are dependent on the concentration of a target analyte of said one or more analytes; and
one or more processors in communication with the optical module and being configured to:
   cause light from the at least one light source to be transmitted towards, and/or through, the at least one sensor layer;
   obtain, from the at least one light detector, one or more optical signals reflected and/or transmitted from the at least one sensor layer; and
   determine, from at least one wavelength component of the one or more optical signals, a target analyte concentration.

### BRIEF DESCRIPTION OF THE DRAWINGS

Some embodiments of a wearable sweat sensor, in accordance with present teachings will now be described, by way of non-limiting example only, with reference to the accompanying drawings in which:

FIG. 1 is a schematic cross-sectional view of a first embodiment of a wearable sweat sensor;

FIG. 2 is a schematic cross-sectional view of a second embodiment of a wearable sweat sensor;

FIG. 3 is a schematic cross-sectional view of a third embodiment of a wearable sweat sensor;

FIG. 4 is a block diagram of a prior art pulse oximeter suitable for use as part of certain embodiments;

FIG. 5 is a system-level block diagram of a wearable sweat sensor according to certain embodiments;

FIG. 6 is a schematic illustration of the working principle of a wearable sweat sensor according to certain embodiments;

FIG. 7 is a block diagram showing data flows and processing steps implemented by a wearable sensor according to certain embodiments;

FIG. 8 is a series of graphs showing performance of a polyaniline film suitable for use with certain embodiments;

FIG. 9 shows transmission characteristics of the polyaniline film;

FIG. 10 shows IR/red signal ratios as a function of pH;

FIG. 11 shows pH v. IR/red ratio for on-body trials of a wearable sensor according to certain embodiments;

FIG. 12 shows graphs from real-time continuous monitoring of pH, heart rate and SpO₂ of four participants in a study conducted using a wearable sensor;

FIG. 13 shows cumulative distribution functions of pH errors for the four study participants;

FIG. 14 shows pH errors for study participants having two different skin types; and

FIG. 15 depicts pairing of a wearable sweat sensor to a smartphone.

### DETAILED DESCRIPTION

Embodiments of the present invention relate to a wearable sweat sensor that incorporates at least one sensor layer for detecting one or more analytes in sweat. The at least one sensor layer is optically coupled to at least one light source and at least one light detector. The at least one sensor layer has optical absorbance properties that are dependent on the concentration of a target analyte of said one or more analytes. Light transmitted by the at least one light source and reflected from the at least one sensor layer can be detected by the at least one light detector, and different wavelength components (for example, infrared and red components) of the detected signal can be measured. When the material of the sensor layer comes into contact with sweat on the surface of the skin of a user and its optical properties change as a result of changes in concentration of the target analyte (such as hydrogen ions, cortisol or glucose), the change in the ratios of the wavelength components can be used to infer the target analyte concentration, for example from a calibration curve that relates the ratio or ratios to pH value, cortisol concentration or glucose concentration.

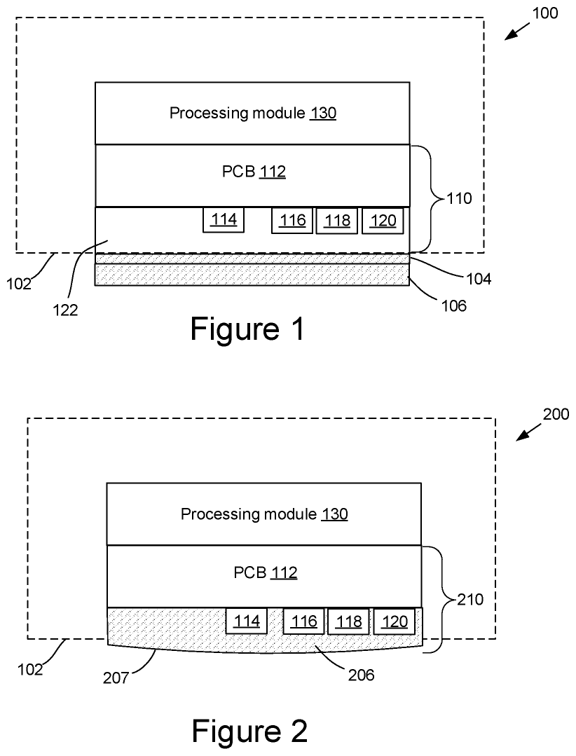

Referring initially to FIG. 1, in a first example, a wearable sweat sensor 100 comprises an optical module 110 that includes a support, such as a PCB 112, to which a plurality of light sources 116, 118 and 120 are attached. The light sources emit at different wavelengths. For example, the light sources may comprise an infrared LED 116, a red LED 118, and a green LED 120. It will be appreciated that fewer or more light sources may be provided as part of the optical module 110. For example, the optical module 110 may comprise just an infrared LED 116 and red LED 118.

A light detector 114, such as a photodiode, is also attached to PCB 112. It will be appreciated that additional detectors may be provided. For example, each light source may have a detector associated with it, and respective detectors may be configured to detect only light emitted from their respective light sources, for example through the use of suitable bandpass filters (such as interference filters).

In some embodiments, a single, broadband, light source may be provided as part of the optical module 110, and the optical module 110 may comprise multiple detectors that are configured to selectively detect different wavelengths, or may comprise a single detector, e.g. an optical spectrometer.

The light sources 116, 118 and 120, and the light detector 114, are protected by a transparent protective layer 122, which may comprise a packaging layer, and optionally, further protection in the form of a glass layer.

PCB 112 includes other components, not illustrated in FIG. 1, such as signal acquisition and signal processing circuitry, data storage, and an interface for connecting the PCB 112 to external devices. In this regard, the wearable sensor 100 can also include a processing module 130 that connects to circuitry of the optical module 110, for example over an I²C interface.

Processing module 130 may comprise a driver to send control signals to the light sources 116, 118 and 120 over the I²C interface, one or more additional communications interfaces (such as a Bluetooth interface) for communicating with external devices, and a data acquisition component to retrieve data from the storage component of optical module 110 for analysis by one or more processors, as will later be described in more detail.

The optical module 110 and processing module 130 may be partly or entirely contained in an external housing 102. The housing 102 may facilitate attachment of the sensor 100 to a user. For example, the housing 102 may be attachable to, or may have integrated as part thereof, a band, strap or clip (not shown) for attachment to the user.

Disposed on protective layer 122, so as to be optically coupled with the light sources and the detector, is a sensor layer 106. For example, the sensor layer may be a layer of a pH-sensitive polymer. In one example, the pH-sensitive polymer may be polyaniline (PANI). PANI has been found to be particularly suitable for pH sensing, as will be shown later with reference to experimental results obtained using an embodiment of a wearable sensor. PANI is also biocompatible.

In other embodiments, the sensor layer 106 may comprise another biocompatible material (such as a polymer) that changes its absorbance properties in response to changes in concentration of a specific target analyte, such as a glucose-responsive hydrogel. In yet further embodiments, the sensor layer 106 may comprise a substrate, such as a polymer (e.g. PDMS) substrate, to which aptamer-conjugated gold nanoparticles are bound, with the aptamer being capable of binding specifically to the target analyte (e.g. cortisol).

The sensor layer 106 may be supported on another layer 104, in particular a flexible layer such as a PDMS layer. This may have several benefits, including facilitating fabrication of the layer 106 and/or attachment of layer 106 to the protective layer 122, and improving skin contact and flexible conformity when the sensor 100 is worn by a user. The sensor layer 106 or support layer 104 may be attached to the protective layer 122 of the optical module 110 in any suitable fashion, for example by use of a transparent adhesive (not shown).

In some embodiments, the optical module 110 may be, or may comprise, a pulse oximeter of the type commonly found in wearable devices such as fitness trackers and smart watches. In such embodiments, therefore, existing devices adapted for certain types of measurements such as heart rate and SpO₂ can be repurposed to act as a sweat sensor (in addition to their existing measurement capabilities). This can be done by affixing the sensor layer 106 to the existing device, and augmenting or replacing software components (e.g. stored on the processing module 130) to compute (for example) pH value, without modifying any internal hardware components of the existing device. To this end, the properties of the sensor layer 106 can be tuned to the emission wavelengths of the light sources of the optical module 110 and/or to the target analyte. For example, sensor layer 106 may be doped (e.g. with various dopant acids) to adjust the peak positions in the absorption spectrum of the layer material such that they are more closely matched to the emission peaks of the light sources.

Another example of a wearable sweat sensor 200 is shown in FIG. 2. Parts in FIG. 2 identical to those in FIG. 1 are assigned the same reference numerals as in FIG. 1, and detailed descriptions thereof are omitted. As can be seen in FIG. 2, the sensor 200 comprises an optical module 210 in which the light sources 116, 118 and 120, and the light detector 114, are encapsulated in a protective layer 206. In this example, the protective layer 206 is itself formed by the sensor layer, such as a pH-sensitive polymer. Accordingly, sensor layer 206 acts as both a protective layer and a sensing layer. This enables the sensor 200 to have a thinner form factor. Additionally, an outer surface 207 of the sensor layer 206 may have a structure that enhances its optical properties. For example, outer surface 207 may be curved such that the polymer layer 206 acts as a lens. In other embodiments, outer surface 207 may have surface structuring, such as a diffractive structure, so that the polymer layer 206 may function as a diffractive optical element (DOE). In either case, the structure and/or shape of outer surface 207 may be such that light is focused towards light detector 114, for example.

As for the example of FIG. 1, the optical module 210 may contain more or fewer light sources and/or light detectors than depicted in FIG. 2, optionally with bandpass filters and the like, to enable different wavelength components of light reflected and/or transmitted from polymer layer 206 to be detected.

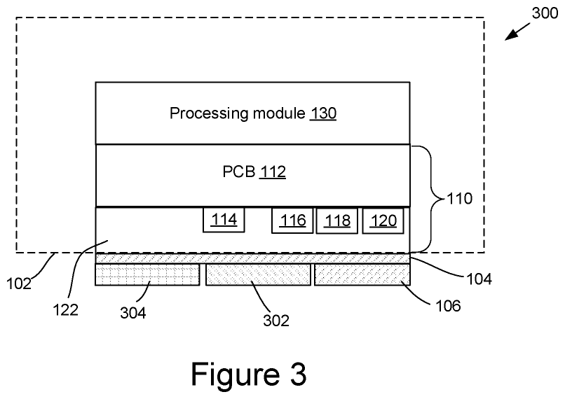

A further example of a wearable sweat sensor 300 is shown in FIG. 3. Parts in FIG. 3 identical to those in FIG. 1 are assigned the same reference numerals as in FIG. 1, and detailed descriptions thereof are omitted.

The sensor 300 may be identical in all respects to the sensor 100 of FIG. 1, except that the sensor layer now includes a plurality of regions 106, 302, 304, that are responsive to different respective analytes. Region 106 is formed from a pH-sensitive polymer such as polyaniline, and may be doped to adjust its absorbance peak(s), as discussed above.

Region 302 is formed from a different material which is sensitive to a different target analyte, such as glucose. For example, a glucose-responsive hydrogel can be incorporated as the material of layer 302. The hydrogel may be based on poly-acrylamide, N,N′-methylenebisacrylamide polymerized with a phenylboronic acid, 3-(acylamido)phenylboronic acid. Inside of the hydrogel, gold nanoparticles with an absorbance peak 600 nm may be introduced. Glucose binding with phenylboronic acid will result in physical swelling of the hydrogel, which will lead to a different aggregation stage of AuNPs. Thus, in the presence of different glucose concentration, the optical absorbance will be different.

Region 304 may be formed from yet another different material which is sensitive to a further target analyte, such as cortisol (a steroid hormone closely related to stress and low blood-glucose concentration). Instead of the hydrogel strategy used for glucose, nucleic acid probes may be immobilised on a PDMS substrate, and then bound with AuNP-modified aptamers which are complementary with the probe. Accordingly, region 304 may comprise PDMS with aptamer-conjugated gold nanoparticles bound thereto. The sweat with cortisol will result in conformational changes of the aptamer and release AuNPs, changing the localized concentration of AuNPs and thus the absorbance intensity. The absorbance intensity will have a direct relation to cortisol concentration in sweat solution. A similar principle applies for other analytes for which aptamers can be designed (by methods known to those skilled in the art), such that other analytes may be detected by sensor layers (or regions thereof) having suitable aptamer-conjugated nanoparticles, such as aptamer-conjugated gold nanoparticles.

As will be appreciated, when more than one analyte is desired to be detected, the optical module 110 may be modified to have additional light sources that emit at different wavelengths, and the sensor regions 106, 302, 304 of the sensor layer may be tuned (for example, using different types or concentrations of dopants such as acids, nanoparticles, etc.) such that their absorbance peaks coincide with or closely match the emission peaks of the various light sources, to facilitate detection of the different analytes.

As depicted in the cross-sectional view of FIG. 3, the regions 106, 302, 304 targeted to different analytes are in side-by-side arrangement. It will be appreciated that the regions targeted to different analytes may be arranged in any suitable fashion in the plane of the sensor layer. For example, patches or strips of the various analyte-specific materials may be interleaved with each other and tiled across the plane of the sensor layer.

The regions 106, 302, 304 are depicted in FIG. 3 as being separated by an air gap. It will be appreciated that other ways of separating the regions are possible, such as providing optically transparent barriers between the regions, the barriers being unreactive with any biomolecular components of human sweat. The barriers may be formed from PDMS, for example.

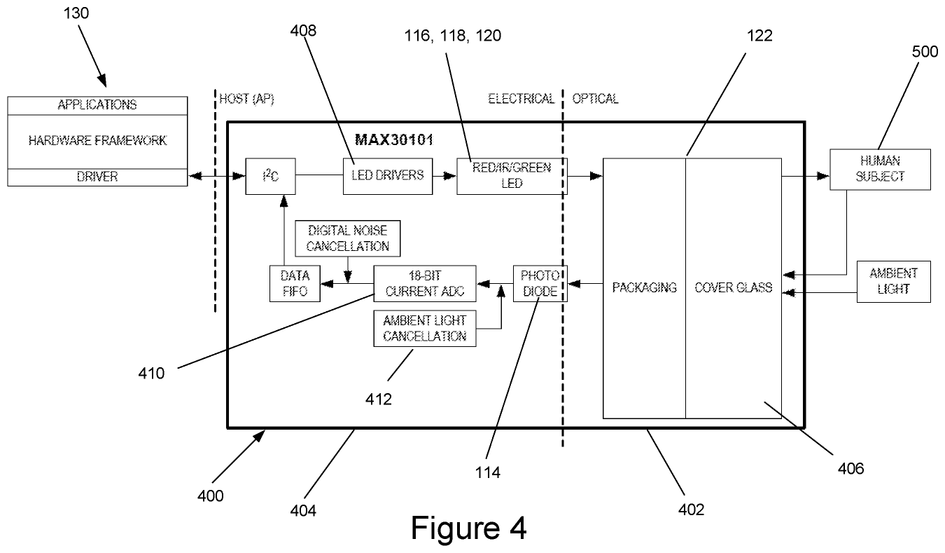

As mentioned above, optical module 110 may be (or may comprise) a standard, off-the-shelf pulse oximeter, such as a MAX30101 high sensitivity pulse oximeter 400 of Maxim Integrated Products, Inc., the block architecture of which is shown in FIG. 4. The Maxim MAX30101 chip 400 is a reflectance LED-based sensor which obviates the need for probes for transmission light sensing, thus enabling small footprint, ultra-low power operation and robust motion artifact resilience. It has been adopted in various wearable hardware, such as the OpenHAK kit and the Hexiwear platform. The chip has two major blocks 402 and 404, one optical, and the other electrical. The optical part 402 integrates cover glass 406 for optimal and robust performance. The electrical subsystem 404 integrates red (peak at 660 nm) 116 and infra-red (peak at 880 nm) 118 LEDs to emit light, with LED drivers 408 modulating the LED pulses for Sp0₂ measurements. The photodiode 114 perceives reflective visible light as well as invisible light and converts the light to an electrical signal proportional to the light intensity. The following 18-bit current ADC (analog-to-digital converter) 410 samples and converts the signal to digitized code with an ambient light cancellation (ALC) function 412. The signal contains the periodicity information of a pulse rate, known as a photoplethysmogram (PPG). The ALC 412 has an internal track/hold circuit to cancel ambient light noise from the reflectance light and improve dynamic range. The Maxim 30101 system 400 also contains an on-chip temperature sensor (not shown) to calibrate for temperature variations.

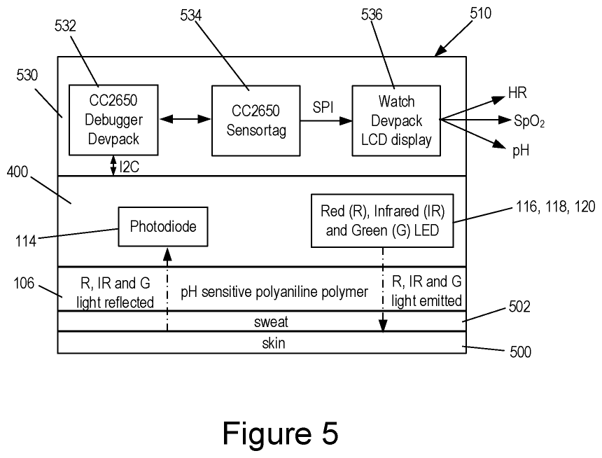

A specific example of a wearable sensor 510 that comprises such a pulse oximeter is depicted in FIG. 5. In the sensor 510 of FIG. 5, the processing module 530 may be, or may comprise, an off-the-shelf module such as the CC2650STK sensortag of Texas Instruments Inc. As such, processing module 530 may include not only a microcontroller and applications software for driving the optical module 400 and receiving and analysing data therefrom, but may also itself include additional sensors, such as accelerometers, gyroscopes, temperature and humidity sensors, etc. For example, as shown in FIG. 5, in addition to a microcontroller 534 (which may integrate or be in communication with sensors such as an inertial measurement unit or a GPS unit), the processing module 530 may comprise a debugger module 532, and a display 536 for displaying values of heart rate, SpO₂ and pH.

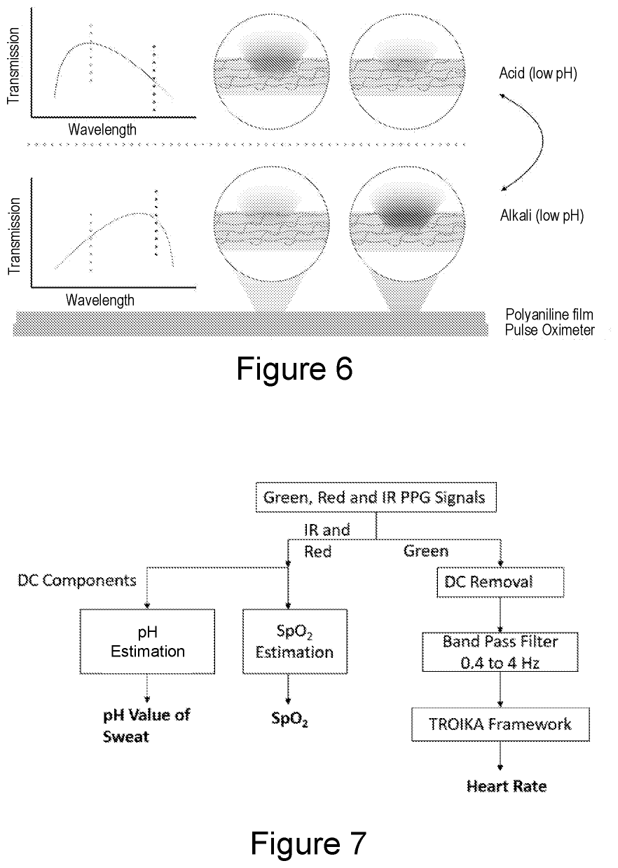

As shown in FIG. 5, in use, the sensor 510 is attached to a user, for example by a band or clip, such that the pH-sensitive polymer layer 106 contacts the user's skin 500. As a layer of sweat 502 forms on the user's skin 500, the optical properties of the polymer (PANI) layer 106 change, due to the pH of the sweat 502. In particular, as illustrated schematically in FIG. 6, when the PANI layer 106 is in contact with acidic (low pH) solution, the polymer becomes protonated, through the conversion of Emeraldine Base (EB) to Emeraldine Salt (ES). This protonation leads to a change in the optical properties of the polymer, and results in strong transmission of light at 680 nm and strong absorbance of light at 880 nm. When in contact with an alkaline (high pH) solution, the polymer becomes deprotonated. In this state, the PANI 106 strongly absorbs light at 660 nm and transmits most of the light at 880 nm. By measuring the relative transmission of light at these two wavelengths, the sensor 510 can be used for real-time pH measurement of sweat.

The sensors 100, 200, 510 each comprise one or more processors in communication with the optical module 110, 210 or 400. At least one of those processors is part of processing module 130 or 530, and is configured to cause light from light sources 116, 118, 120 to be transmitted towards the layer 106 of the pH-sensitive polymer. For example, processing module 130 or 530 may comprise a microcontroller (MCU) that is used to drive the light sources, e.g. over an I²C interface as shown in FIG. 4. The MCU may be configured to send sequences of control signals to cause pulsing of the light sources in a particular sequence at a desired sampling rate for a desired duration. For example, the MCU may be configured to cause optical module 110, 210 or 400 to sequentially emit pulses of light from red LED 116 and infrared LED 118 for SpO₂ measurements. The signals detected by photodiode 114 in response to those pulses may also be used to determine pH, as will be described below.

During each sampling period, the MCU also receives data over the I²C interface indicative of optical signals detected by the light detector 114. The data includes signals corresponding to different wavelengths (e.g., red and infrared). Once sampling has been conducted for the desired duration, another processor of processing module 130/530, or even a processor of an external device with which processing module 130/530 is in communication (e.g., over a Bluetooth connection), may then determine, from the two different wavelength components (red and infrared) of the optical signals, a pH level of the user.

An example software architecture implemented by a wearable sweat sensor is depicted in FIG. 7. The software architecture, and the processes performed by the wearable sweat sensor, will be described below by reference to the MAX30101 pulse oximeter and CC2650 sensortag embodiment of FIG. 5, but it will be appreciated that it may be readily adapted for other optical modules and/or processing modules.

In the sensor 510, all the software algorithms for pH, heart rate and Sp0₂ estimation are performed on the CC2650 sensortag 534 for every time window of 5 seconds. The number of samples for processing is constrained at M=FₛT/4=100×5/4=125 samples due to the small SRAM in CC2650. However, it is also possible to process more samples with a faster CPU and increase the processing time window T. The software flow of sensor 510 shown in FIG. 7 is as follows:

(1) Optical module (MAX30101) 400 shines red, infrared and green light on the wrist with sweat and reads the reflected PPG signals and sends the ADC values of the PPG signals over I²C bus to processing module (CC2650) 530 for 2 seconds. Accelerometer readings from CC2650 530 are also read for the same 2 seconds. The samples are read and stored in 6 KB sensor controller in CC2650 530.

(2) Green PPG Signals are preprocessed with DC removal and band pass filtering. Accelerometer readings are also band pass filtered. Then, heart rate is calculated using the TROIKA framework for motion artifact removal. A description of TROIKA is contained in Zhang et al., IEEE Trans Biomed Eng. 2015 February; 62(2):522-31, the entire contents of which are hereby incorporated by reference.

(3) Infrared and red PPG signals are read, DC and AC components extracted and SpO₂ values calculated.

(4) Finally, DC components of IR and red PPG signals are used to calculate the pH value of sweat.

(5) All the calculated pH value, Heart rate and Sp0₂ values are shown on Watch Devpack LCD display 536 (FIG. 5).

(6) Repeat step (1).

The first two seconds of PPG signals and intermediate values are flushed immediately after computation. The source code in CC2650 spans about 800 lines and takes about 4 seconds for execution.

Before beginning to exercise or be involved in any activity that stimulates sweating, the user may be prompted to press the user button in CC2650 which records the average of DC components of infrared and red PPG signals reflected from wrist without sweat.

The pH, heart rate and SpO₂ determination operations will now be described in more detail.

Determination of pH Value

When MAX30101 400 mounted with PANI 106 is placed on the wrist 500 with sweat 502, the reflected PPG signals comprise 5 components. The DC component of PPG signals come from four components—reflection from tissue and sweat, reflection from non-pulsatile arterial blood, pulsatile arterial blood, and reflection from PANI film 106. The AC component of the PPG signal comes from the pulsatile arterial blood. Therefore, reflected IR and Red components from the PANI film 106 corresponding to pH value of sweat is present in the DC component of the signal. Also, a major portion of the DC component (about 80%) of the PPG signal is contributed by the reflection from tissue. Therefore, we need to separate the DC component reflected from PANI from the overall DC component of the IR and Red PPG signals.

We define time series PPG signal recorded by sensor 510:

I=[I(0),I(1), . . . ,I(M−1)], R=[R(0),R(1), . . . ,R(M−1)]

where I and R are the reflected infrared and red PPG signals respectively recorded by sensor 510. M is the number of samples. The sensor 510 is configured to operate at a sampling frequency fₛ=100 Hz, sample averaged by 4 since MAX30101 chip 400 can send data by averaging adjacent samples to reduce throughput and we calculate pH for every sliding time window of T=5 seconds with overlap of S=3 seconds. So, M=fₛT=125 samples. DCI and DCR are the DC components of infrared and red PPG signal respectively. Infrared and red LEDs are switched on alternately in MAX30101 400 to avoid self heating and reduce power consumption. Although IR and Red pulse repetition frequency can go up to 100 KHz, we chose 100 Hz as the sampling rate since low sampling frequency will also reduce power consumption.

Our goal is to find the DC component reflected from PANI 106 that corresponds to the pH value of sweat, given by DC_{I}^{PANI} and DC_{R}^{PANI} from DC_{I} and DC_{R} respectively. DC_{I} and DC_{R} are calculated by finding the mean of I and R PPG signals recorded by sensor 510 respectively given by

$$D {C}_{I} = \frac{1}{M} {\sum }_{n = 0}^{M - 1} I ( n ) and D {C}_{R} = \frac{1}{M} {\sum }_{n = 0}^{M - 1} R ( n )$$

In order to find DC_{I}^{PANI} from the DC component of the PPG signal, we need to first separate the reflected DC component from tissue and pulsatile as well as non-pulsatile arterial blood denoted as [symbol] which cannot be calculated deterministically. However, the DC components reflected from the skin, tissue, fat, bones etc. remain constant over time. Also, the blood volume in the veins and capillaries remains constant and thus, reflected DC components of PPG signals are constant over time. So, before starting pH measurements, we initialize the [symbol] as the average of DC components measured from the wrist without sweat for 3 seconds. At the start of real-time pH sensing, since there will not be any sweat on the skin for PANI sensor 106 to react with, we will not consider the pH of skin. This average of reflected DC components measured from wrist devoid of sweat for 3 seconds is denoted by DC_{I}^{init} and DC_{R}^{init} for infrared and red respectively. Using the initialized values, we can DC_{I}^{PANI} by subtracting DC^{init} from the DC component of the PPG signal for each time window. Therefore, we have

DC_{I}^{PANI}=DC_{I}−DC_{I}^{init},DC_{R}^{PANI}=DC_{R}−DC_{R}^{init}

From this, IR/Red ratio can be calculated as:

$$IR / Red ratio = \frac{D {C}_{I}^{PANI}}{D {C}_{R}^{PANI}}$$

Using the calculated IR/Red ratio, we can use a pH versus IR/Red ratio calibration (for which see below) and calculate the pH of sweat for every time window of 5 seconds. The above pH estimation approach does not suffer from motion artifacts caused by hand swing since typical frequency of motion artifacts is in between 0.5 Hz-5 Hz. This pH estimation approach was evaluated with on-body trials on 10 participants (see later), showing an accuracy of about 91% in estimating pH with error of ±0.6 pH in real time.

It will be appreciated that a procedure similar to the above may be used to determine concentrations of other types of target analyte (with, for example, a previously derived calibration curve of target analyte concentration v. ratio of signals at different wavelengths being used for the computation).

Heart Rate Sensing

Pulse oximeters shine red, infrared and green light on the skin and measure heart rate (HR) from the reflected green photoplethysmograph (PPG) signals. For heart rate measurements, we use green PPG signals instead of red or IR PPG signals because the wrist has less blood perfusion and a higher energy PPG signal (green light) is preferable. The MAX30101 has three LEDs, namely red, infrared, and green. As explained earlier, we use the IR and red PPG signals for sweat pH estimation. For heart rate monitoring, in addition to the IR and red PPG signals, we also use the green PPG signals.

PPG signals are greatly affected by motion artifacts (MA) which interfere with the measurement of heart rate. Acceleration data has been shown to be an effective solution for removing MA from PPG signals even during intense physical exercise. The CC2650 sensor tag is equipped with an accelerometer, so we made use of both the accelerometer readings from CC2650 sensor tag and the green PPG signals obtained from MAX30101 to remove MA from PPG signal using the light-weight TROIKA framework. TROIKA consists of three main parts—(a) Signal decomposition (b) Sparse Signal Reconstruction and (c) Spectral Peak tracing.

In the TROIKA framework, PPG signals are processed in sliding time windows of T seconds with incremental steps of S seconds, preferably S≥T/2 and HR is estimated at each time window of T seconds. We chose T=5 seconds and S=2 seconds.

Before feeding the PPG signals into the TROIKA framework, the following preprocessing is done on the signal:

(1) DC removal is performed on the PPG Signal to achieve a baseline detrended signal with zero mean

(2) The PPG signal and 3-axis accelerometer data are band pass filtered between 0.4 and 5 Hz to remove noise and MA lying outside the heart rate frequency.

It has been found that preprocessing and TROIKA MA removal is able to successfully clean the signal for HR monitoring with the sensor 510. Accordingly, the addition of PANI film 106 to the pulse oximeter 400 does not hinder the ability of the pulse oximeter 400 to measure heart rate even during motion. Therefore, it is believed that when PANI film 106 is mounted on current day fitness trackers, any custom motion artifact removal algorithm (not just TROIKA) developed by the manufacturers of the fitness trackers can also be used to estimate the heart rate even during intense physical exercise.

SpO₂ Estimation

For estimating SpO₂, we need to calculate the ratio R presented in the following equation from the AC and DC components of the Red and Infrared signals respectively.

$$R = \frac{{R}_{red}}{{R}_{infra - red}} = \frac{A {C}_{red} / D {C}_{red}}{A {C}_{infra - red} / D {C}_{infra - red}}$$

In order to handle motion artifacts, we use the cleansed PPG signal obtained using Singular Spectral Analysis (SSA) as the AC component of the IR and red PPG signals. For SpO₂ measurements, we calculate the ratio R between infrared and red PPG signals as defined immediately above. The scattering constant and the optical path length vary considerably between the red and infrared wavelengths. Thus, the calculated ratio R cannot be directly related to the physiological oxygen saturation (SpO₂). Therefore the relationship between ratio R and the physiological SpO₂ for any commercial pulse oximeter is determined experimentally through calibration. For any pulse oximeter, R is determined for many healthy volunteers using the pulse oximeters and their SpO₂ is measured by collecting their arterial blood. Using the obtained R and SpO₂ values, a curvilinear approximation or empirical calibration of pulse oximeter is made to obtain a more precise SpO₂ measurement. In sensor 510, the standard calibration equation recommended by MAX30101, SpO₂=104-17R, is used to estimate the SpO₂ value.

Fabrication Methods, Performance Measurements and pH Calibration

Sensor fabrication. To improve skin contact and flexible conformity, we functionalized PANI on a supportive, flexible and transparent matrix prepared with polydimethylsiloxane (PDMS). The PDMS support was prepared by mixing the elastomer with the curing agent at a weight ratio of 10:1. The mixture was poured onto a master mould, and cured in an oven at 75° C. for 1 h. Following curing, the PDMS layer is peeled from the master. The PDMS replica (2 mm height) was thoroughly washed with isopropanol. To functionalize the surface of PDMS 104 with PANI film 106, we first exposed the PDMS surface to oxygen plasma for 60 s (Harrick Plasma). The activated PDMS was incubated with a 20% wt solution of N-[3(trimethoxylsilyl)propyl]aniline in ethanol for 60 mins. Using this technique, a monolayer of silane-bearing aniline was formed on the substrate via molecular self-assembly. Chemical deposition of the PANI on the surface of PDMS was performed by immersing the PDMS with freshly prepared 1 M HCl solution containing the oxidant (0.25 M ammonium peroxydisulfate) and 1 M aniline. The pendant aniline on the surface served as the initiation site for polymerization and was also used to covalently anchor the PANI film 106 on the substrate 104. The polymerization time was fixed to 1 day. After polymerization, the PDMS-PANI were washed extensively with water to remove any unattached PANI. The resulting films had good adhesion due to the chemical bonding between the substrate and polymer film.

The fabrication cost of our PANI-PDMS sensor 104, 106 is less than $1. Furthermore, owing to the process simplicity and the applicability for various shape and size casts, our technique allows for mass fabrication of our pH sensor.

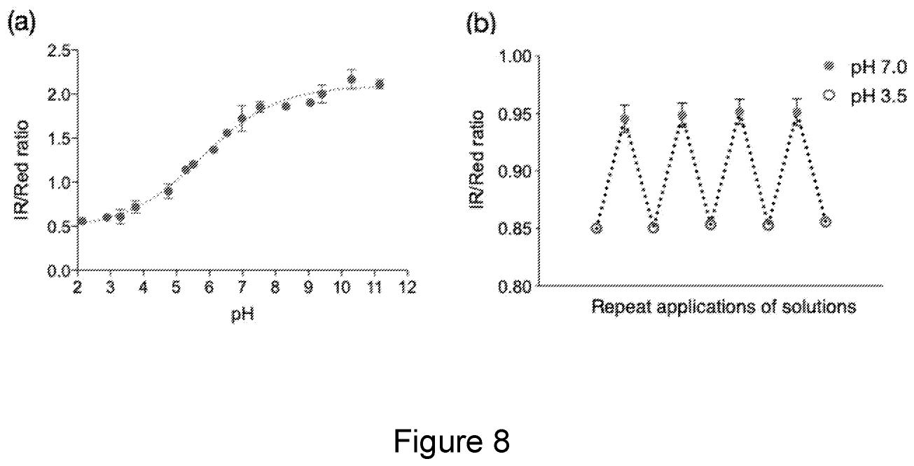

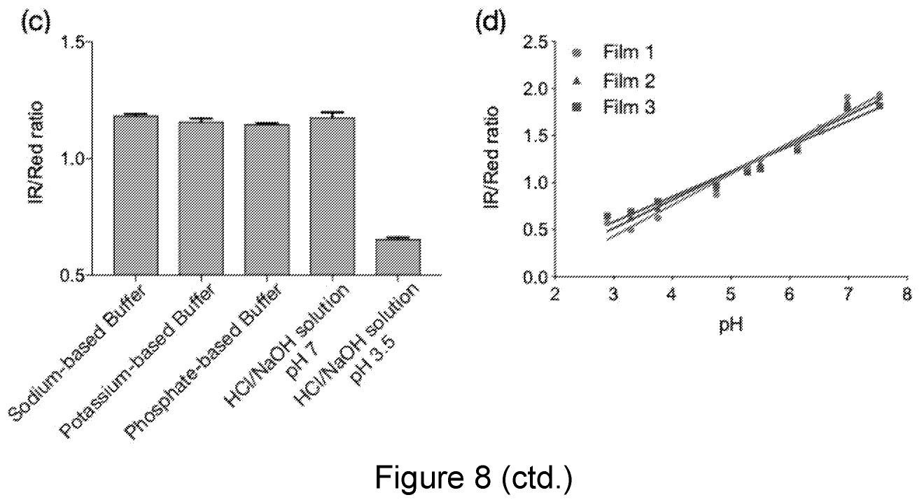

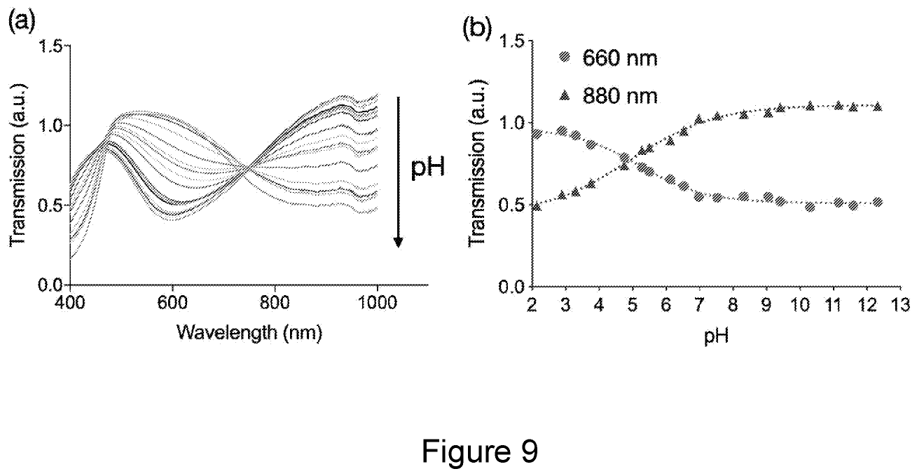

Sensor performance measurements. We first evaluated the optical transmission spectra of the prepared PANI films using a plate reader. A plate reader is a device that emits light at specific wavelengths on the PANI film 106 and measures the absorbance/transmission from the reflected light. Films were incubated in different pH solutions for 1 min before collection of the UV-Vis transmission spectra. As depicted in FIG. 9(a), the PANI optical changes are highly sensitive to pH changes. As the pH was increased from 2 (acidic) to 12 (alkaline), the PANI film showed a shift in its absorption peak from 420 nm (at pH 2) to 605 nm (at pH 12). This change is consistent with published studies on the different degree of protonation of the imine nitrogen atoms in the polymer chain. We next plotted the pH dependence of the transmission at 660 nm and 880 nm, respectively (FIG. 9(b)). The characteristic PANI sigmoid shape curves were obtained with good correlations against a wide range of pH changes, R2 (660 nm)=0.989 and R2 (880 nm)=0.996. To further improve the detection accuracy and the material stability, we obtained the ratio of the transmission intensity at 880 nm to the transmission intensity at 660 nm, as shown in FIG. 8(a). As we varied the solution pH from 2 to 12, this intensity ratio decreased and saturated. It is worth noting that this ratio change was most significant in the range of pH 3-8, which is also the physiological pH fluctuation range of human sweat. To demonstrate the repeatability of the sensor for measuring pH changes, we tested the integrated system repeatedly by using two different pH solutions, pH 3.4 and pH 7.0 prepared through mixing different amounts of HCl and NaOH solutions. As shown in FIG. 8(b), after changing the pH value of the solution alternately for 4 cycles, PANI film maintained a relatively consistent IR/Red ratio at the same pH, indicating the repeatability of this material. The sensor not only demonstrated good reproducibility, to generate compatible signal outputs when treated with solutions of the same pH, but also showed excellent responsiveness and repeatability. Because body sweat contains a variety of ions such as Na⁺, K⁺, Cl⁻, P⁵⁻ and H⁺, we next tested the specificity of the integrated sensor to pH changes amidst a complex ionic background. We chose four different solution mixtures, namely sodium-based buffer, potassium-based buffer, phosphate-based buffer and HCl/NaOH solution (pH 7), respectively. As these solutions contain varying concentration of above mentioned ions (Na⁺, K⁺, Cl⁻, P⁵⁻ and H⁺); these mixtures were prepared to pH=7. Aside from these solutions, we also prepared a mixture which is HCl/NaOH solution (pH 3.5). We incubated the PANI film with these solutions for 1 minute and ratiometric measurements were performed. As demonstrated in FIG. 8(c), the sensor is selectively responsive to pH changes. We finally tested the reproducibility of the film preparation in generating reliable analysis. We prepared multiple PANI films and measured the signal ratios of these sensors when incubated in various pH solutions. As shown in FIG. 8(d), the sensors yielded highly uniform and robust signals, without demonstrating any significant deviations during the entire pH monitoring with a very small relative standard deviation (RSD) of 5.3%.

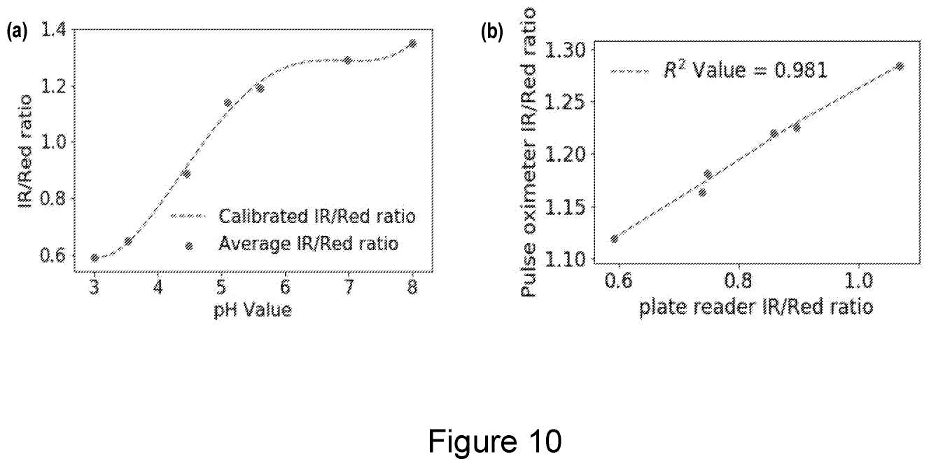

pH calibration. For sensing pH value from sweat, we need to first calibrate the reflected IR to Red ratio from (for example) MAX30101 mounted with PANI for different pH values. For calibrating IR/Red Ratio curve using MAX30101, we created synthetic sweat solutions with pH value between 3 and 8 since pH of human sweat lies between 3 and 8. We placed 200 μL of each pH solution on MAX30101 and measured the average of the reflected IR to Red ratio for 30 seconds. We repeated the procedure for 3 times for each pH solution. For each pH value, we used the average of IR/Red ratios recorded during 3 different trials and calibrated the pH versus IR/Red ratio curve by performing a 4th order polynomial fit of the recorded IR/Red ratios for each pH solution. We used a 4th order polynomial fit instead of linear fit because PANI does not produce IR/Red ratios in a linear trend as seen in FIG. 8(a) and polynomial fit produces more accurate results than linear fit. FIG. 10(a) shows the calibrated pH versus IR/Red ratio curve clearly showing the same increasing trends as the pH versus IR/Red ratio curve in FIG. 8(a) obtained using plate reader. We also performed an ordinary least squares (OLS) fit between the IR/Red ratios obtained from plate reader and IR/Red ratios recorded from MAX30101. The resulting fit shown in FIG. 10(b) has a very high R² value of 0.981 indicating that the IR/Red ratios obtained from MAX30101 follow the same trend as the IR/Red ratios obtained using plate reader.

Experimental Evaluation

Accuracy of pH-Watch on Synthetic pH Solutions

Firstly, we evaluated MAX30101 mounted with PANI without placing it on skin to compute the accuracy of PANI in estimating pH using calibration curve in FIG. 10(a). We created synthetic buffer solutions of different pH value by mixing different concentrations of HCl and NaOH. We used a commercial wireless pH meter “HI14142—HALO® Wireless pH Meter” from Hannah Instruments for measuring the pH value of each solution. HI14142 is also compatible with measuring pH value of sweat directly from the skin. We placed 200 μL of each solution on PANI and measured the pH values from the reflected IR/Red ratios. Table 2 shows the pH value of different solutions measured by commercial pH meter and pH value measured by MAX30101 mounted with PANI.

TABLE 2
pH of synthetic solutions measured
by commercial pH meter and pH-Watch

| pH value of | Estimated | |
|---|---|---|
| the Solution | pH Value | % error |
| 3.54 | 3.45 | 2.5 |
| 4.46 | 4.57 | 2.5 |
| 4.98 | 4.87 | 2.2 |
| 5.5 | 5.63 | 2.4 |
| 6.13 | 6.22 | 1.8 |
| 6.98 | 6.84 | 2.0 |
| 7.54 | 7.43 | 1.5 |

| | |
|---|---|
| Average error (%) | 2.13% |

The average error between the pH measured by commercial pH meter and estimated pH value was found to be 2.13%. We can see that the estimated pH value varies from the commercial pH meter readings by at most 2.5% which is comparable to <2.2% pH variations in a PANI based electrochemical pH sensor reported in the literature.

Accuracy of pH-Watch in Heart Rate (HR) and SpO₂ Measurements

We evaluated the accuracy of pH watch in heart rate and SpO₂ measurements with and without motion artifacts. We recruited a participant who wore the pH watch on his left wrist and MAX30102 high sensitivity finger pulse oximeter (with IR and Red LED) on his right hand serving as a ground truth for HR and SpO₂ measurements. The measurements from MAX30102 were measured and logged using the MAX30102ACCEVKIT evaluation board which reads in the finger PPG measurements and calculates HR and SpO₂. During the experiment, we restrained the participant from moving his right hand so that the ground truth HR and SpO₂ values are not affected by MA. Each experiment was carried out for 120 seconds. The experiment was repeated 8 times without motion in the left hand with sensor 510 for testing accuracy without motion and 8 times with participant being asked to induce random motion in left hand continuously by hand swings and heavy to and fro shaking of left hand, for testing accuracy in the presence of MA. For experiments with motion in left hand, we asked the user to stay stationary for first 3 seconds, since TROIKA requires initialization of heart rate. Finally, for each experiment we compared the resulting HR and SpO₂ measurements of sensor 510 against the ground truth HR and SpO₂ measurements recorded by finger pulse oximeter.

Accuracy of HR and SpO2 Measurements without Motion Artifacts.

Table 3 shows HR measurements recorded by sensor 510 without hand motion validated against finger pulse oximeter readings which serves as ground truth. Average percentage error of HR and SpO2 measurements were 1.22% and 2.75% respectively with HR and SpO₂ varying by a maximum of <3.44% and <3.22% respectively. The errors observed were negligible and may be attributed to the fact that PPG readings from finger are inherently more accurate than PPG readings from wrist due to the better blood perfusion in fingers.

TABLE 3
HR and SpO₂ measurements

| | HR | | | SpO₂ | | |
|---|---|---|---|---|---|---|
| | (BPM) | | | (%) | | |
| | pH | MAX | | pH | MAX | |
| Data | Watch | 30102 | % Error | Watch | 30102 | % Error |
| 1 | 85 | 86 | 1.16 | 92.5 | 95 | 2.63 |
| 2 | 82 | 84 | 2.38 | 91 | 94 | 3.2 |
| 3 | 85 | 83 | 2.35 | 94 | 97 | 3.1 |
| 4 | 83 | 82 | 1.2 | 96 | 98 | 2.04 |
| 5 | 74 | 75 | 1.33 | 93 | 95.5 | 2.61 |
| 6 | 83 | 84 | 1.19 | 94 | 96 | 2.1 |
| 7 | 84 | 87 | 3.44 | 95 | 98 | 3.06 |
| 8 | 73 | 73 | 0 | 91 | 93 | 3.22 |

| | | | |
|---|---|---|---|
| Average HR | 1.22 | Average SpO₂ | 2.75 |
| % Error | | % Error | |

Accuracy of HR and SpO2 Measurements with Motion Artifacts.

Table 4 shows HR measurements recorded by sensor 510 with random hand motions validated against finger pulse oximeter readings serving as ground truth. Average percentage error of HR and SpO₂ measurements were 4.98% and 4.57% respectively with HR and SpO₂ varying by a maximum of <6.41% and <6.73% respectively. This is comparable to the average heart rate error of 1.8% with maximum variation of 4.70% as reported originally with TROIKA. The small difference in error rate is due to the fact that sensor 510 is configured to use very small time windows of 5 seconds owing to RAM limitation in CC2650. The study in the original TROIKA paper used time windows of 10 seconds with 1250 samples and hence could have more accurate HR measurements than pH Watch. The average errors could be further reduced with a more powerful CPU with higher RAM, which most current day wearables already have. For instance, the latest Samsung Galaxy watch has a 1.15 GHz Exynos 9110 processor with 768 MB RAM. Thus, the experiments clearly show that pH Watch with PANI does not hinder the normal ability of pulse oximeters in measuring HR and blood oxygen concentration, thereby confirming that our pH sensing approach could be easily integrated with all current-day smart watches or fitness trackers with the addition of our PANI film 106.

TABLE 4
HR and SpO₂ measurements

| | HR | | | SpO₂ | | |
|---|---|---|---|---|---|---|
| | (BPM) | | | (%) | | |
| | pH | MAX | | pH | MAX | |
| Data | Watch | 30102 | % Error | Watch | 30102 | % Error |
| 1 | 83 | 88 | 5.68 | 93.5 | 98 | 4.59 |
| 2 | 82 | 78 | 4.87 | 92 | 97 | 5.15 |
| 3 | 81 | 86 | 5.8 | 93 | 96 | 3.2 |
| 4 | 80 | 76 | 5.26 | 89 | 94 | 5.32 |
| 5 | 79 | 75 | 5.33 | 91 | 95 | 4.21 |
| 6 | 78 | 81 | 3.7 | 90 | 96.5 | 6.73 |
| 7 | 70 | 72 | 2.78 | 92 | 94.5 | 2.12 |
| 8 | 73 | 78 | 6.41 | 90 | 95 | 5.26 |

| | | | |
|---|---|---|---|
| Average HR | 4.98 | Average SpO₂ | 4.57 |
| % Error | | % Error | |

On-Body Trials

To evaluate the accuracy and efficiency of pH watch in real-time monitoring of pH from human sweat, we recruited 10 participants and conducted on-body-trials with sensor 510 for measuring their sweat pH value.

For the first experiment to test the accuracy of sensor 510 in sensing pH value from sweat, we recruited 6 participants. Each participant was given a randomized ID and the data collected from them where mapped to the randomized ID to protect their personal data. Each participant was asked to wear sensor 510 and exercise for 10-20 minutes so that they start perspiring. Before beginning to exercise, the tissue DC of the participants given by DC^{init} were measured for 3 seconds by pressing the user button in sensor 510. Once the participants started to sweat, we compared the pH value of sweat measured by sensor 510 against the pH value of sweat by means of a HI14142 wireless pH meter to calculate the accuracy. The above procedure is repeated twice for each participant.

In the second experiment to assess the capability of sensor 510 for real-time continuous sensing of pH value and detection of dehydration during exercise, we recruited 4 participants who wore sensor 510 and cycled in the gym for 80 minutes. The HR, SpO₂ and pH values measured by pH watch were logged. During exercise, we measure the ground truth pH value manually using wireless pH meter every 5 or 10 mins due to the limited output rate of our wireless pH meter. Our wireless pH meter takes around 30 seconds to give a stable reading. This does not affect our evaluations because pH of sweat changes very slowly with exercise and does not change considerably within 10 mins.

Our evaluations took into consideration a variety of skin tones, from Chinese skin types to moderately dark skinned Indian people between 21-30 years of age (Chinese, Malays and Indians). All our participants were males. A summary of skin types and demographics of the participants is shown in Table 6.

TABLE 6
Skin characteristics of participants in trials

| | | | | Number of |
|---|---|---|---|---|
| | | | | participants |
| | | | | who took part |
| Skin | Skin | Number of | Age | in continuous |
| Type | Color | participants | range | sensing |
| Chinese/ | Light- | 5 | 21-30 | 2 |
| Malay | skinned | | | |
| Indian | Light- | 3 | 24-27 | 2 |
| | brown | | | |
| Indian | Dark- | 2 | 24-27 | 0 |
| | brown | | | |

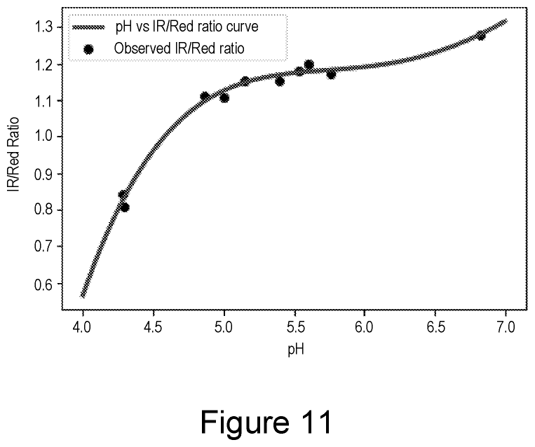

Accuracy of pH Watch. Table 7 shows pH measurements made by pH watch validated against the pH value of sweat measured by wireless pH meter during on-body trials by following the first experimental procedure. Average percentage error was 2.31% from the pH meter readings with the maximum variation of <4.3%. We also plotted the pH versus IR/Red ratio curve for pH values we measured during this experiment as shown in FIG. 11 and the curve resembles the calibration curve in FIG. 10(a). We observed a R² value of 0.99 indicating very high correlation between the two curves. This is similar to previous wearable pH sensing approaches showing—(1) maximum variation of <2.2% reported by PANI based electrochemical pH sensing approach and (2) maximum variation of <8.3% (±0.5 pH change) reported by ORMOSIL textile fabric based optical pH sensing approach. This indicates that sensor 510 has sensing accuracy comparable with current state-of-art pH sensors. Yet, it is long-term reusable and compatible with today's pervasive wearables.

TABLE 7
pH measured by pH Watch during on-body trials

| pH value | pH estimated | |
|---|---|---|
| of sweat | by pH Watch | % Error |
| 4.28 | 4.36 | 1.8 |
| 4.29 | 4.33 | 0.9 |
| 4.78 | 4.87 | 1.9 |
| 4.86 | 5.02 | 3.3 |
| 5 | 4.98 | 0.4 |
| 5.15 | 5.35 | 3.8 |
| 5.4 | 5.36 | 0.7 |
| 5.53 | 5.61 | 1.4 |
| 5.6 | 5.82 | 3.93 |
| 5.76 | 5.52 | 4.2 |
| 6.43 | 6.27 | 2.5 |
| 6.82 | 7.01 | 2.8 |

| | |
|---|---|
| Average % Error | 2.31% |

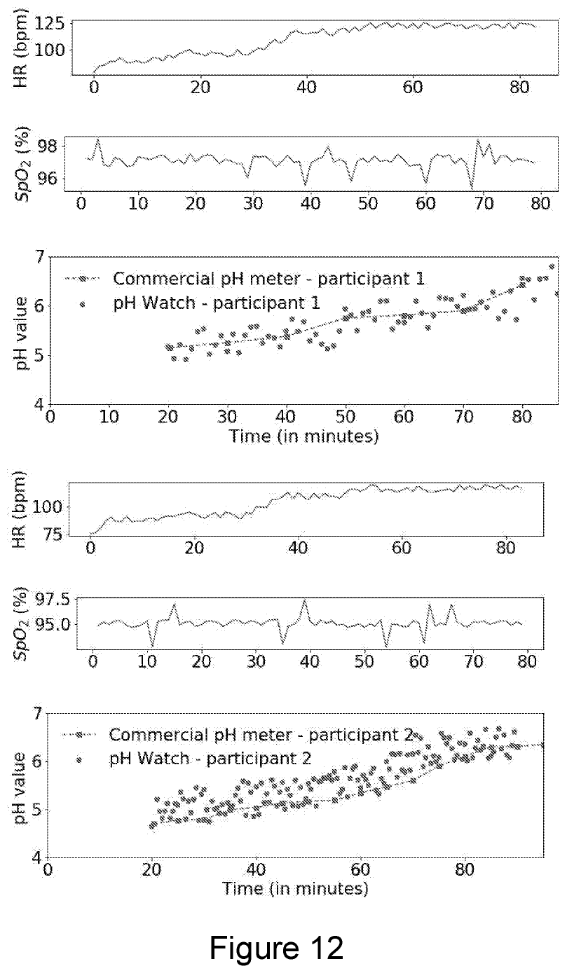

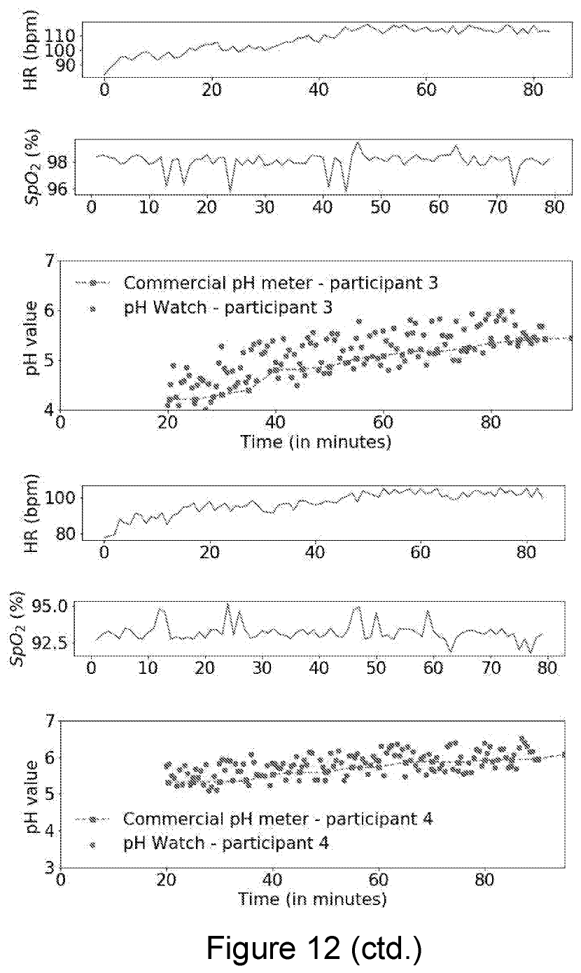

Real-time continuous monitoring of HR, SpO2 and pH. FIG. 12 shows HR, SpO2 and pH measurements of a participant continuously recorded by sensor 510 during the second experimental procedure for assessing the real-time sensing capacity of sensor 510. The real-time heart rate measurements show that:

(1) In the beginning of the exercise, the heart rate spiked from 70 to 90 BPM within the first 5 minutes indicating that the exercise had started.

(2) HR remained almost constant for the next 10 minutes around 90 BPM and again increased to 100 BPM indicating that the exercise is picking up and the participant is cycling faster now.

(3) HR remained around 110 BPM for the next 15 minutes and then increased to 120 BPM and stabilized around 120 BPM. This increase can be due to decrease in hydration levels in human body as perspiration happens during exercise. This drop in hydration levels of human body causes mild strain on the heart since blood volume decreases. Also blood will be carrying more sodium as a result of decreased hydration levels which makes it more difficult for the heart to pump blood. Therefore the heart beats faster resulting in increased HR.

On the other hand, real-time SpO₂ measurements remain consistent, around 97% most of the time with minimum SpO₂ around 96% and maximum SpO₂ around 98%. This agrees well with the fact that SpO₂ levels would regulate themselves and remain stable during low and middle intensity exercise where the person breathes in enough oxygen continuously during exercise.

FIG. 12 shows real time pH values validated against pH values of sweat measured by the wireless pH meter. Real time pH values increase with time during exercise. For the first 20 minutes, the participant did not sweat and hence no pH readings were shown in the graph. It took 20 minutes for the participant to sweat because the wrist region does not sweat so fast. After 20 mins, the participant started to sweat and for the next 20 mins, the pH value of the sweat remained stable around 5.2 to 5.3. This stability in pH value of sweat is because the sweat rate will be very limited during the moderate phase of exercising. Once the user starts to kick in with increasing the intensity of workout, the pH value of sweat rises from 5.3 to 5.7 denoting increase in the sweat rate of the participant. This is also appropriately indicated in the heart rate measurements where there is a clear increase in the heart rate to 120 BPM due to increase in sweat rate and reduction in hydration levels. Then the pH slowly increased from 5.7 to 5.8 for the next 20 minutes and again increased to 6.2 within 10 minutes. This also confirms that, as the person exercises for a prolonged duration (70-80 minutes), his sweat rate increases and also the concentration of sodium in his sweat increases. This in turn, increases the pH value of sweat.

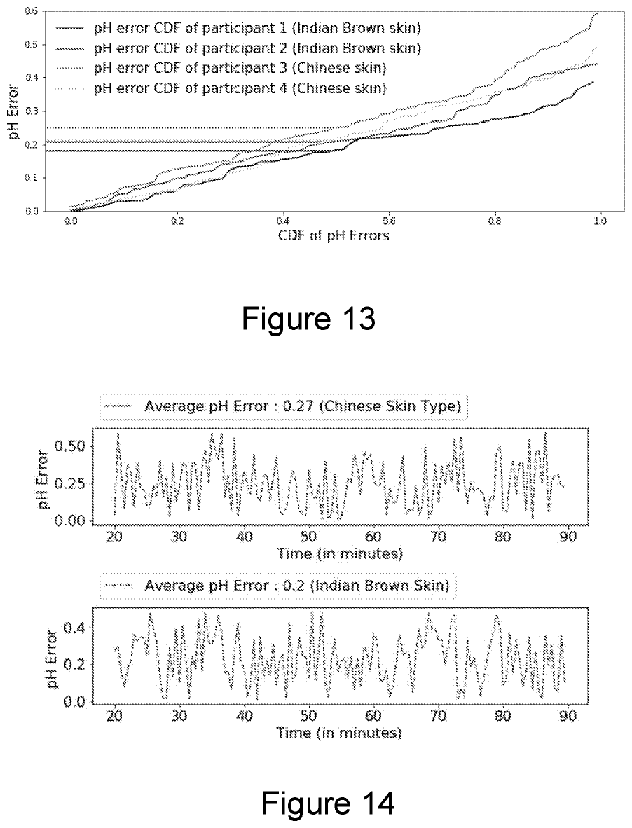

We repeated the same experimental procedure with 3 other participants and the real-time pH, HR and SpO2 values measured by sensor 510 is shown in FIG. 12. FIG. 13 shows the cumulative distribution function of pH errors for each of the 4 participants.

The median pH error for all the participants ranged from 0.2 to 0.28. The maximum pH error ranged from 0.4 to 0.6. Therefore, pH values measured by sensor 510 differed from the wireless pH meter by a maximum of ±0.6 pH change comparable to the results reported previously and also shows that sensor 510 can detect pH with an accuracy of about 91%. This clearly indicates the potential of sensor 510 in detecting dehydration levels in human body during exercise and future research on correlation between heart rate and pH value of sweat could prove to be helpful in detecting an accurate dehydration risk with just the addition of an low cost off-the-shelf pH sensitive PANI 106 to the pulse oximeters being used in current day fitness trackers.

For our real-time continuous sensing experiment, we had incorporated participants of Chinese skin type and Indian skin type. FIG. 14 shows pH errors reported by Indian skin and Chinese skin types. We can observe that Indian skin and Chinese skin had similar average pH errors of 0.2 and 0.27 respectively which validates that our pH measurement shows similar errors for different skin types and remain relatively unaffected by skin colour. More intuitively, skin colour has little effect on our pH measurements, because skin colour contributes to the DC part of the PPG signal which is measured during the initialization part of our pH sensing algorithm and removed during the pH measurement.

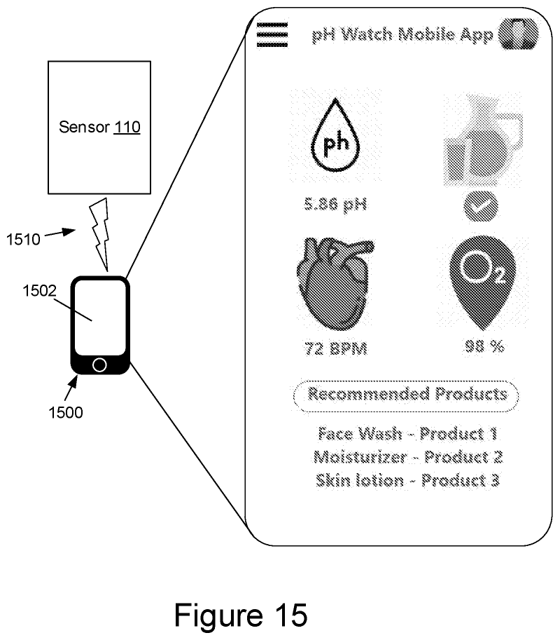

In some embodiments, the sensor 100, 200, 300 or 510 may be configured to pair with an external device such as a smartphone 1500, as depicted in FIG. 15. The pairing may be by way of a Bluetooth connection 1510, for example.

Smartphone 1500 may execute a mobile application (app) that enables a user wearing the sensor (e.g. sensor 510) to continuously track dehydration risk/skin health by monitoring trends in the real-time pH values obtained from the sensor 510.

The mobile app may recommend a skin care regime and cosmetics based on trends observed in the pH value of sweat. The mobile app may also provide a “Drink Water” feature, which generates an alert whenever the user's pH value of sweat rises above a normal range, thereby helping the user to stay hydrated at all times. Normal sweat pH values range between 4.5-7.0. Under dehydration, the sweat pH remains at higher levels of 6-8 (similar to water). The Drink Water feature implemented in the mobile app may remind the user to drink water whenever a pH value greater than 6 is detected by sensor 510. After drinking water and once the user's pH becomes normal again, the mobile app may generate a further notification that hydration levels are sufficient.

An example GUI wireframe for the mobile app, displayable on display 1502 of the mobile device, is shown in the zoomed view at the right of FIG. 15. The GUI enables easier health tracking from the sensor 510.

The sweat pH determined by sensor 510 is an indicator of skin health. It is known that cosmetic products alter the pH of human skin. In order to suggest the right cosmetics for a user's skin, the pH values obtained from sensor 510 can be used to recommend cosmetic products with a suitable corresponding pH value for maintaining skin health and ensuring that cosmetics do not adversely affect the skin pH. To this end, the mobile app may maintain a database of commonly used cosmetic products, and their corresponding pH values.

The use of pH-sensitive polymers in embodiments of the present invention has numerous advantages. These polymers can be readily integrated with wearables due to their flexible nature, ease of miniaturization, and good biocompatibility. In particular, conductive polymer polyaniline (PANI) offers not only large-range and sensitive pH responsiveness, but also enables real-time optical readouts. After proton-mediated post-polymer doping and dedoping, PANI exhibits significant changes in the near-infrared spectrum, which is a target frequency of pulse oximeters.

Mounting a sensor layer such as a pH-sensitive polymer layer on a pulse oximeter to enable real-time, reusable and continuous monitoring of a target analyte in sweat (such as pH) has the following advantages. First, the polymer acts as a dual matrix support and indicator dye which is a substance used to show visually the condition of a solution with respect to the presence of a particular material (such as different concentration of hydrogen ion) by change of color, and can be readily interfaced for safe skin contact. The polymer is biocompatible and does not disintegrate. This improves long-term stability, as it eliminates any possible dye leaching, which is a common problem seen in sensors using other pH-responsive small molecule dyes. Second, when treated with different pH solutions, PANI shows a significant and rapid response at the wavelengths 660 nm and 880 nm—distinct wavelengths illuminated and measured by many pulse oximeters. This high compatibility for direct integration thus enables sensitive and reusable monitoring of sweat pH.

Embodiments can also effectively control the thickness of PANI film, and adapt to the thickness requirements of different equipment. At first, from the macro perspective, it is possible to readily change the thickness of the substrate-PDMS. In addition, for different thickness of the PANI film, it can be controlled by changing temperatures. The films become thicker with increasing temperatures and reach around few microns. The film growth is faster when the temperature is increased and indicate that it accelerated with time. Based on the adjustability of thickness, the polymer film can be adjusted according to the morphology and appearance design of different chips.

Advantageously, in fabricating a PANI layer on PDMS, a mask can be added to PDMS, such that a specific array of silane-bearing aniline was only formed on the exposed substrate via molecular self-assembly. By tuning the exposure area, it is possible to change the transparency of the PANI sensor layer. Through tuning the transparency of PANI film, detection signals become more compatible. For example, under high transmittance of the polymer layer, removing the light absorbed by the PANI itself, the remaining light can be used to detect other targets in other possible modules.

Many modifications will be apparent to those skilled in the art without departing from the scope of the present invention.

Throughout this specification, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

A description of some exemplary embodiments of the present disclosure is contained in one or more of the following numbered statements.
1. A wearable sweat sensor for detecting one or more analytes in human sweat, comprising:
   an optical module comprising at least one light source and at least one light detector attached to a support;
   at least one sensor layer optically coupled to the optical module, the at least one sensor layer having optical absorbance properties that are dependent on the concentration of a target analyte of said one or more analytes; and
   one or more processors in communication with the optical module and being configured to:
      cause light from the at least one light source to be transmitted towards, and/or through, the at least one sensor layer;
      obtain, from the at least one light detector, one or more optical signals reflected and/or transmitted from the at least one sensor layer; and
      determine, from at least one wavelength component of the one or more optical signals, a target analyte concentration.
2. A wearable sweat sensor according to 1, wherein the optical module comprises a pulse oximeter.
3. A wearable sweat sensor according to 1 or 2, wherein the optical module comprises a plurality of light sources that emit light at different wavelengths.
4. A wearable sweat sensor according to any one of 1 to 3, wherein the one or more processors are further configured to determine, from the one or more optical signals, a heart rate and/or SpO₂ of the user, in addition to determining the target analyte concentration.
5. A wearable sweat sensor according to any one of 1 to 4, wherein the optical module comprises a plurality of light detectors configured to detect light at different wavelengths.
6. A wearable sweat sensor according to any one of 1 to 5, wherein the one or more processors are configured to determine the target analyte concentration of the user based on a ratio of two different wavelength components of the one or more optical signals.
7. A wearable sweat sensor according to any one of 1 to 6, wherein one of said analytes is hydrogen ions, and wherein at least one sensor layer comprises a pH-sensitive polymer layer.
8. A wearable sweat sensor according to 7, wherein the pH-sensitive polymer is polyaniline.
9. A wearable sweat sensor according to any one of 1 to 8, wherein one of said analytes is glucose, and wherein at least one sensor layer comprises a glucose-responsive hydrogel.
10. A wearable sweat sensor according to 9, wherein the hydrogel comprises poly-acrylamide, N,N′-methylenebisacrylamide polymerized with 3-(acylamido)phenylboronic acid.
11. A wearable sweat sensor according to 9 or 10, wherein the glucose-responsive hydrogel comprises gold nanoparticles.
12. A wearable sweat sensor according to 11, wherein the gold nanoparticles have an absorbance peak of 600 nm.
13. A wearable sweat sensor according to any one of 1 to 12, wherein the at least one sensor layer comprises a polymer layer having aptamer-conjugated gold nanoparticles bound thereto, said aptamers being capable of binding specifically to the target analyte.
14. A wearable sweat sensor according to 13, wherein the target analyte is cortisol.
15. A wearable sweat sensor according to any one of 1 to 14, comprising a plurality of sensor layers each being configured to detect a different target analyte of said one or more analytes.
16. A wearable sweat sensor according to any one of 1 to 15, wherein at least one of said sensor layers comprises a plurality of regions each being configured to detect a different target analyte of said one or more analytes.
17. A wearable sweat sensor according to any one of 1 to 16, wherein the wavelength components comprise a red component and an infrared component.
18. A wearable sweat sensor according to any one of 1 to 17, wherein the at least one sensor layer comprises a plurality of regions each having different thickness and/or surface texture and/or levels of a dopant.
19. A wearable sweat sensor according to any one of 2 to 18, wherein the at least one sensor layer is attached to, or is integral with, the pulse oximeter.
20. A wearable sweat sensor according to 19, wherein at least one sensor layer forms a protective layer of the pulse oximeter.
21. A wearable sweat sensor according to any one of 1 to 20, comprising one or more of a band, clip, and adhesive layer for attachment of the support to the user.
22. A wearable sweat sensor according to any one of 1 to 21, wherein the one or more processors are configured to generate an alert based on the determined pH level.

1. A wearable sweat sensor for detecting one or more analytes in human sweat, comprising:
an optical module comprising at least one light source and at least one light detector attached to a support;
at least one sensor layer optically coupled to the optical module, the at least one sensor layer having optical absorbance properties that are dependent on the concentration of a target analyte of said one or more analytes; and
one or more processors in communication with the optical module and being configured to:
cause light from the at least one light source to be transmitted towards, and/or through, the at least one sensor layer;
obtain, from the at least one light detector, one or more optical signals reflected and/or transmitted from the at least one sensor layer; and
determine, from at least one wavelength component of the one or more optical signals, a target analyte concentration.

2. A wearable sweat sensor according to claim 1, wherein the optical module comprises a pulse oximeter.

3. A wearable sweat sensor according to claim 1, wherein the optical module comprises a plurality of light sources that emit light at different wavelengths.

4. A wearable sweat sensor according to claim 1, wherein the one or more processors are further configured to determine, from the one or more optical signals, a heart rate and/or SpO₂ of the user, in addition to determining the target analyte concentration.

5. A wearable sweat sensor according to claim 1, wherein the optical module comprises a plurality of light detectors configured to detect light at different wavelengths.

6. A wearable sweat sensor according to claim 1, wherein the one or more processors are configured to determine the target analyte concentration of the user based on a ratio of two different wavelength components of the one or more optical signals.

7. A wearable sweat sensor according to claim 1, wherein one of said analytes is hydrogen ions, and wherein at least one sensor layer comprises a pH-sensitive polymer layer.

8. A wearable sweat sensor according to claim 7, wherein the pH-sensitive polymer is polyaniline.

9. A wearable sweat sensor according to claim 1, wherein one of said analytes is glucose, and wherein at least one sensor layer comprises a glucose-responsive hydrogel.

10. A wearable sweat sensor according to claim 9, wherein the hydrogel comprises poly-acrylamide, N,N′-methylenebisacrylamide polymerized with 3-(acylamido)phenylboronic acid.

11. A wearable sweat sensor according to claim 9, wherein the glucose-responsive hydrogel comprises gold nanoparticles.

12. A wearable sweat sensor according to claim 1, wherein the at least one sensor layer comprises a polymer layer having aptamer-conjugated gold nanoparticles bound thereto, said aptamers being capable of binding specifically to the target analyte.

13. A wearable sweat sensor according to claim 12, wherein the target analyte is cortisol.

14. A wearable sweat sensor according to claim 1, comprising a plurality of sensor layers each being configured to detect a different target analyte of said one or more analytes.

15. A wearable sweat sensor according to claim 14, wherein at least one of said sensor layers comprises a plurality of regions each being configured to detect a different target analyte of said one or more analytes.

16. A wearable sweat sensor according to claim 1, wherein the wavelength components comprise a red component and an infrared component.

17. A wearable sweat sensor according to claim 1, wherein the at least one sensor layer comprises a plurality of regions each having different thickness and/or surface texture and/or levels of a dopant.

18. A wearable sweat sensor according to claim 2, wherein the at least one sensor layer is attached to, or is integral with, the pulse oximeter.

19. A wearable sweat sensor according to claim 18, wherein at least one sensor layer forms a protective layer of the pulse oximeter.

20. A wearable sweat sensor according to claim 1, comprising one or more of a band, clip, and adhesive layer for attachment of the support to the user.
